(19) United States
(12) Patent Application Publication
(10) Pub. No.: US 2024/0299606 A1
(43) Pub. Date: Sep. 12, 2024

(54) DISINFECTION MODULE FOR A SEMI-AUTONOMOUS CLEANING AND DISINFECTION DEVICE

(71) Applicant: Avidbots Corp, Kitchener (CA)

(72) Inventors: Pablo Roberto MOLINA CABRERA, Waterloo (CA); Ravi BABOOLAL, Cambridge (CA); Thomas GRANDIN, Kitchener (CA); Alan MAGNI, Kitchener (CA); Dan CORNIES, Kitchener (CA); Justin MCCOMBE, Kitchener (CA)

(21) Appl. No.: 18/058,726

(22) PCT Filed: Apr. 30, 2021
(86) PCT No.: PCT/CA2021/050611

§ 371 Date: Nov. 23, 2022

Related U.S. Application Data
(60) Provisional application No. 63/055,919, filed on Jul. 24, 2020.

(30) Foreign Application Priority Data
Aug. 12, 2020 (WO) PCT/CA2020/051100

(51) Int. Cl. A61L 2/22; A61L 2/18; A61L 2/24

(52) U.S. Cl. CPC A61L 2/22; A61L 2/186; A61L 2/24; A61L 2202/14; A61L 2202/15; A61L 2202/16; A61L 2202/17; A61L 2202/25

(57) ABSTRACT

An autonomous or semi-autonomous cleaning device having a disinfection module mounted therein for disinfecting walls and objects in the areas it operates. The disinfection module consists of a fan, atomizer nozzle, an electrostatic module and a disinfection tank to store disinfection solution. The nozzle of the disinfection module will spray a stream of disinfection solution towards walls and objects to disinfect these surfaces. The system activates an electrostatically charged disinfection misting system which is designed to disinfect vertical surfaces and select horizontal surfaces.

### CROSS REFERENCE TO RELATED APPLICATIONS

The application is a National Phase application that claims priority to and the benefit of the International Application Serial No. PCT/C2021/050611 entitled “DISINFECTION MODULE FOR A SEMI-AUTONOMOUS CLEANING AND DISINFECTION DEVICE”, filed on Apr. 30, 2021, which claims the benefit of U.S. Provisional Patent Application Ser. No. 63/055,919, entitled “DISINFECTION MODULE FOR A SEMI-AUTONOMOUS CLEANING AND DISINFECTION DEVICE”, filed on Jul. 24, 2020; the disclosures of which are incorporated herein by reference in their entirety.

### BACKGROUND

The embodiments described herein relate to semi-autonomous cleaning and disinfection devices and more particularly, to a system and method for a disinfection module for a semi-autonomous cleaning device for cleaning of surfaces.

There is a problem in the janitorial industry where business facilities must quickly adopt a quality disinfection solution while easing the risk and burden of their human cleaning staff. This problem is particularly notable where surfaces may become contaminated during an opening period, and need to be disinfected when a facility re-opens. Facilities managers are currently spending substantial additional effort having cleaning staff manually disinfect more frequently. Manual cleaning efforts are prone to error, and often miss sections of surfaces where disinfection is required.

Due to the COVID-19 and other infectious diseases, certain locations will be required to consistently disinfect common areas in order to control the spread and then avoid a disease transmission. Facilities such as hospitals and airports are looking at traditional solutions such as manual scrubbing or spraying walls, objects, artifacts, and facilities managers expect audits that demonstrate that their facilities are cleaned and disinfected on a regular basis.

The most common pattern identified now is related to the need to disinfect touch surfaces. This can take up to 70% of a cleaning staff's time during COVID-19. Some of those steps are done by personnel by scrubbing or spraying manually, some are done with larger sprayers with larger effective coverage areas. Spraying seems the most practical solution long term for the following reasons: it does not require confining the space, it does not require personal protective equipment (like fogging), it remains fast (compared to solutions like ultraviolet (UV) that require 5-10 minutes exposure), and is compliant with the safety regulations for the use of disinfecting chemicals.

Cleaners of public spaces have a mandate to clean more stringently in the wake of the COVID-19 virus (i.e., increase the frequency of cleaning) and have a daily depleting workforce (i.e., labor shortage) to do that job. An autonomous or semi-autonomous cleaning robotic device that can also assist in cleaning and disinfection of surfaces is described herein.

### SUMMARY

An autonomous or semi-autonomous cleaning device having a disinfection module mounted for disinfecting walls and objects in the areas it operates. The disinfection module consists of a fan, atomizer nozzle, an electrostatic module and a disinfection tank to store disinfection solution. The nozzle of the disinfection module sprays a stream of disinfection solution towards walls and objects to disinfect these surfaces. The system activates an electrostatically charged disinfection misting system which is designed to disinfect vertical surfaces and select horizontal surfaces.

### BRIEF DESCRIPTION OF THE DRAWINGS

FIG. 1 is a perspective view of a semi-autonomous cleaning device.

FIG. 2 is a front view of a semi-autonomous cleaning device.

FIG. 3 is a back view of a semi-autonomous cleaning device.

FIG. 4 is a left side view of a semi-autonomous cleaning device.

FIG. 5 is a right-side view of a semi-autonomous cleaning device.

FIG. 6 is a perspective view of a semi-autonomous cleaning device with an external disinfection module.

FIG. 7 is a perspective view of a semi-autonomous cleaning device discharging a disinfection solution.

FIG. 8A is a perspective view of a semi-autonomous cleaning device displaying an internal disinfection module tank.

FIG. 8B is a right-side view of a semi-autonomous cleaning device displaying an internal disinfection module tank.

FIG. 9 is a top plan view of a semi-autonomous cleaning device displaying an external disinfection module tank.

FIG. 10 is a further perspective view of a semi-autonomous cleaning device with another external disinfection module design.

FIGS. 11A to 11E are line drawing illustrating a semi-autonomous cleaning device with another external disinfection module design.

FIG. 12 is a diagram illustrating key components of a disinfection module.

FIGS. 13A and 13B are diagrams illustrating the control system.

FIGS. 14A-14C are diagrams illustrating the sprayer module.

FIGS. 15A and 15B are diagrams illustrating the sprayer head of the sprayer module.

FIGS. 16A and 16B are diagrams illustrating the tank and pump module.

FIG. 17 is a line drawing illustrating a perspective view of components of a disinfection module.

FIGS. 18A to 18E are diagram illustrating graphical user interfaces (GUI) for the disinfection module.

### DETAILED DESCRIPTION

This disclosure focuses on providing an extra spraying system to provide autonomous and semi-autonomous cleaning devices to disinfect walls and objects in the areas it operates. The goal is to automate and scale disinfection in public spaces in order to gain consistency at a fraction of the current cost.

That ideal solution should quickly and effectively disinfect facilities, ease the burden of manual disinfection by human labor, and meet any existing or anticipated disinfection requirements in the wake of businesses reopening. It should also be innovative yet competitive, cost effective and flexible enough to allow for a level of adaptation to the moment so that the facility manager can manage risks associated with pandemic uncertainty. Data should also be collected on which surfaces have been disinfected.

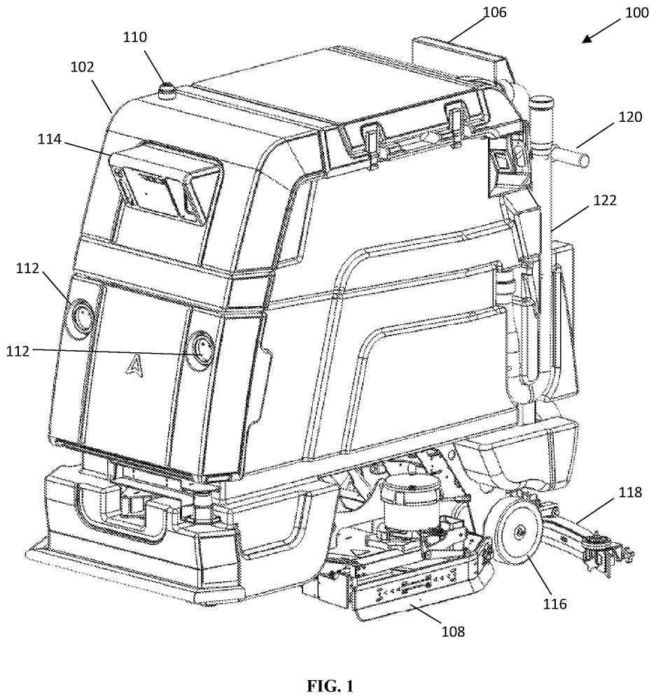

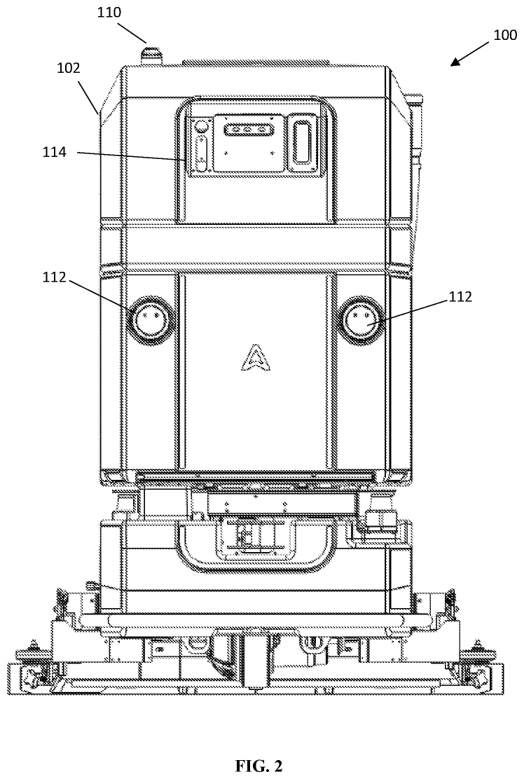

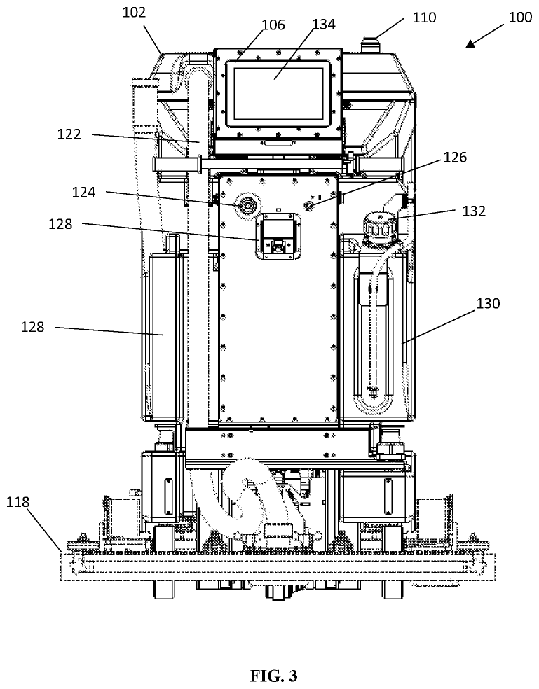

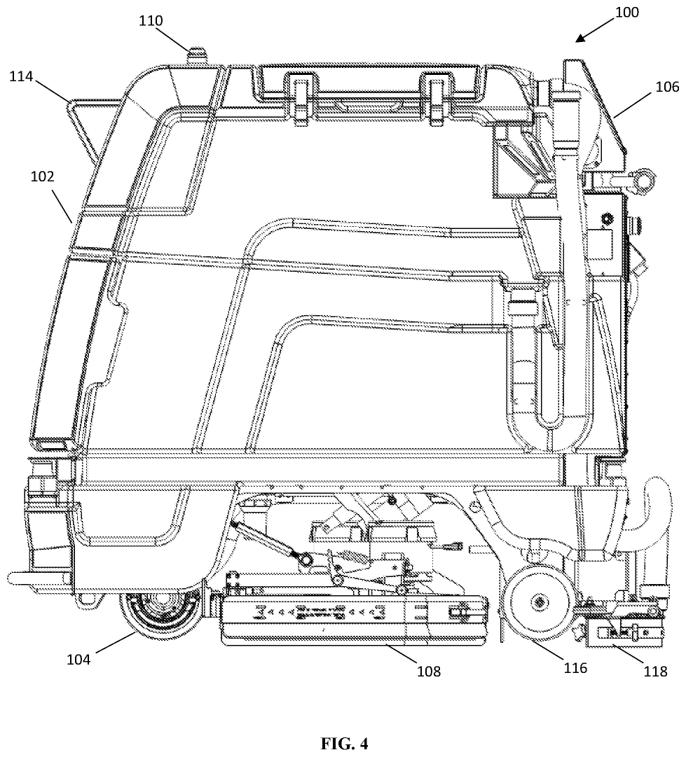

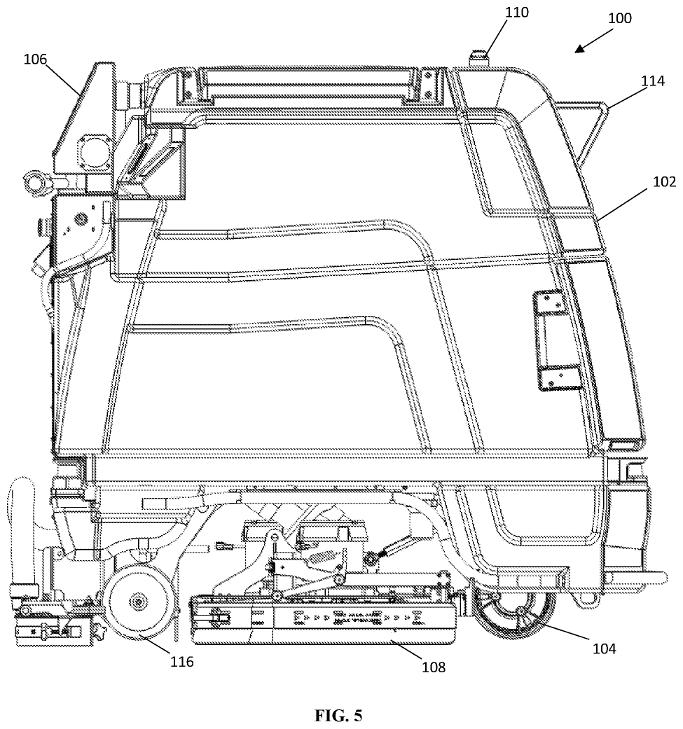

An exemplary embodiment of a semi-autonomous cleaning device as shown in FIG. 1 is a perspective view of a semi-autonomous cleaning device. FIG. 2 is a front view of a semi-autonomous cleaning device. FIG. 3 is a back view of a semi-autonomous cleaning device. FIG. 4 is a left side view of a semi-autonomous cleaning device, and FIG. 5 is a right-side view of a semi-autonomous cleaning device.

FIGS. 1 to 5 illustrate a semi-autonomous cleaning device 100. The device 100 (also referred to herein as “cleaning robot” or “robot”) includes at least a frame 102, a drive system 104, an electronics system 106, and a cleaning assembly 108. The cleaning robot 100 can be used to clean (e.g., vacuum, scrub, disinfect, etc.) any suitable surface area such as, for example, a floor of a home, commercial building, warehouse, etc. The robot 100 can be any suitable shape, size, or configuration and can include one or more systems, mechanisms, assemblies, or subassemblies that can perform any suitable function associated with, for example, traveling along a surface, mapping a surface, cleaning a surface, and/or the like.

The frame 102 of cleaning device 100 can be any suitable shape, size, and/or configuration. For example, in some embodiments, the frame 102 can include a set of components or the like, which are coupled to form a support structure configured to support the drive system 104, the cleaning assembly 108, and the electronic system 106. Cleaning assembly 108 may be connected directly to frame 102 or an alternate suitable support structure or sub-frame (not shown). The frame 102 of cleaning device 100 further comprises strobe light 110, front lights 112, a front sensing module 114 and a rear sensing module 128, rear wheels 116, rear skirt 118, handle 120 and cleaning hose 122. The frame 102 also includes one or more internal storage tanks or storing volumes for storing water, disinfecting solutions (i.e., bleach, soap, cleaning liquid, etc.), debris (dirt), and dirty water. More information on the cleaning device 100 is further disclosed in PCT publication WO2016/168944, entitled “APPARATUS AND METHODS FOR SEMI-AUTONOMOUS CLEANING OF SURFACES” filed on Apr. 25, 2016 and International Application Serial No. PCT/CA2020/051100, entitled “SYSTEM AND METHOD OF SEMI-AUTONOMOUS CLEANING OF SURFACES” filed on Aug. 12, 2020, the disclosures of which are incorporated herein by reference in their entirety.

More particularly, in this embodiment, the front sensing module 114 further includes structured light sensors in a vertical and horizontal mounting position, an active stereo sensor and an RGB camera. The rear sensing module 128, as seen in FIG. 3, consists of a rear optical camera. In further embodiments, front and rear sensing modules 114 and 128 may also include other sensors including one or more optical camera, thermal cameras, LiDAR (Light Detection and Ranging), structured light sensors, active stereo sensors (for 3D) and RGB cameras.

The back view of a semi-autonomous cleaning device 100, as seen in FIG. 3, further shows frame 102, cleaning hose 122, clean water tank 130, clean water fill port 132, rear skirt 118, strobe light 110 and electronic system 106. Electronic system 106 further comprises display 134 which can be either a static display or touchscreen display. Rear skirt 118 consists of a squeegee head or rubber blade that engages the floor surface along which the cleaning device 100 travels and channels debris towards the cleaning assembly 108.

FIG. 3 further includes emergency stop button 124 which consists of a big red button, a device power switch button 126 and a rear sensing module 128. Rear sensing module 128 further comprises an optical camera that is positioned to sense the rear of device 100. This complements the front sensing module 114 which provides view and direction of the front of device 100, which work together to sense obstacles and obstructions.

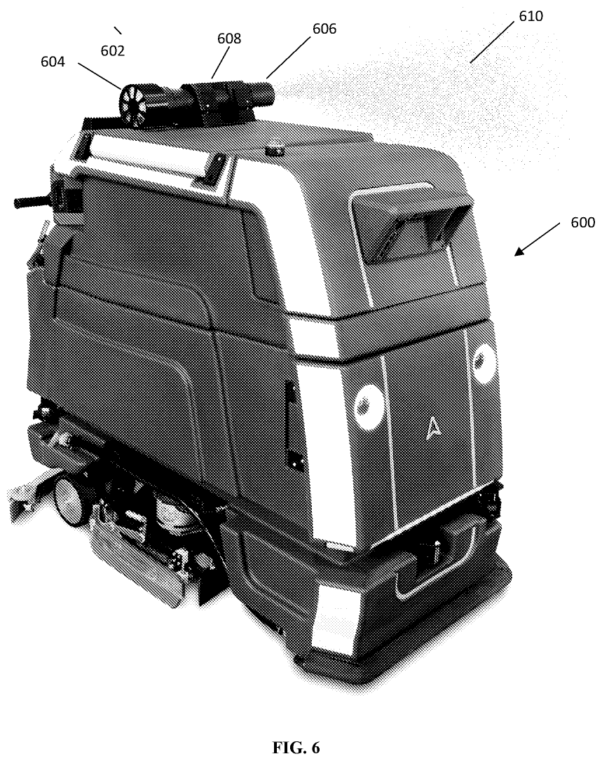

FIG. 6 is a perspective view of a semi-autonomous cleaning device with an external disinfection module 602. As seen in FIG. 6, components of the disinfection module comprise of:
High powered fan (DC voltage) 604
Atomizer nozzle 606
Electrostatic module cathode
Electrostatic module 608

According to FIG. 6, the disinfection module consists of a spraying, misting and/or fogging system that will distribute a disinfectant solution 610 onto touch areas such as handles, doors, handrails, touchscreens, tables, countertops, shelves, and other areas that need regular disinfecting. The disinfection module mounts to a semi-autonomous cleaning device and is able to automatically navigate to any location of the facility and disinfect it as needed using the automation and infrastructure of the existing product.

The disinfection module 602 may contain a solution tank, an atomizing system, a dispersion system, and an electrostatic system. The system will be mounted so the disinfectant solution 610 can spread at an appropriate height and within a 1.5 m distance from the cleaning device. By utilizing an electrostatic system, the module can maximize total coverage and disinfectant despite spray angle. Further info on the disinfection module can be found in the U.S. provisional application No. 63/055,919, entitled “DISINFECTION MODULE FOR A SEMI-AUTONOMOUS CLEANING AND DISINFECTION DEVICE”, filed on Jul. 24, 2020, which is incorporated herein by reference in its entirety.

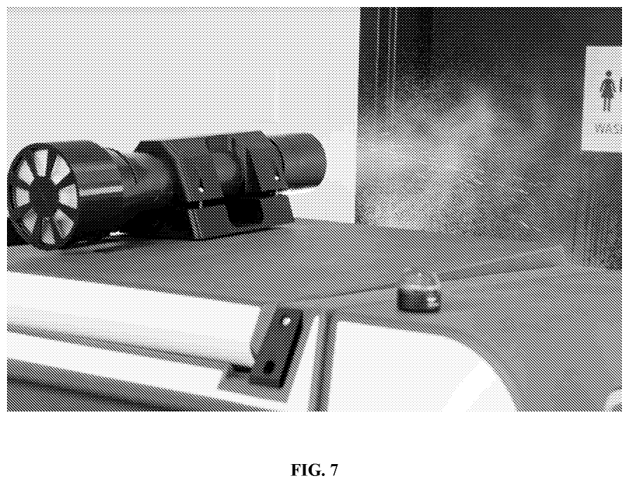

FIG. 7 is a perspective view of a semi-autonomous cleaning device discharging a disinfection solution. As seen in FIG. 7, the function of the disinfection module is to spray an electrostatically charged disinfection solution into an airstream that then travels to vertical/horizontal surfaces. These surfaces may include washroom doors, handles, doorknobs, walls and entryways.

According to this disclosure, the disinfection solution would be any chemical that can be sprayed, commonly but not limited to quaternary ammonium, hydrogen peroxide, iso-propyl, and any other chemical or liquid that can be sprayed and recognized for having disinfecting properties. Furthermore, the disinfection solution may contain perfume, room fresheners and odor killing solutions (i.e., Febreze®).

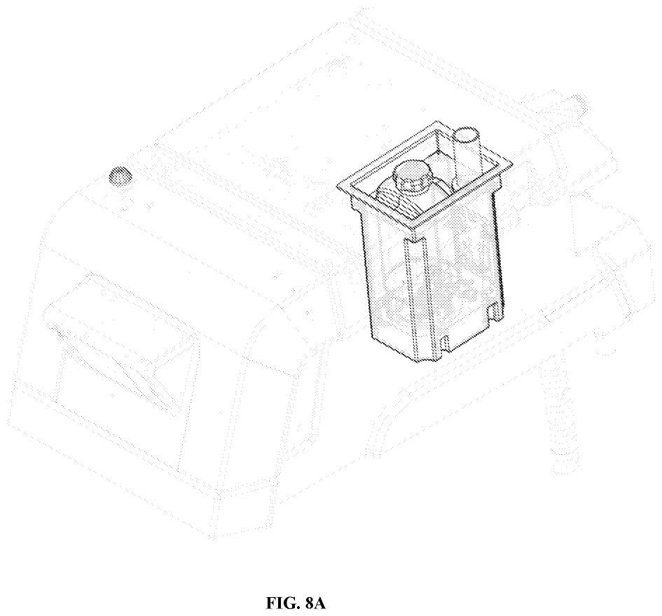

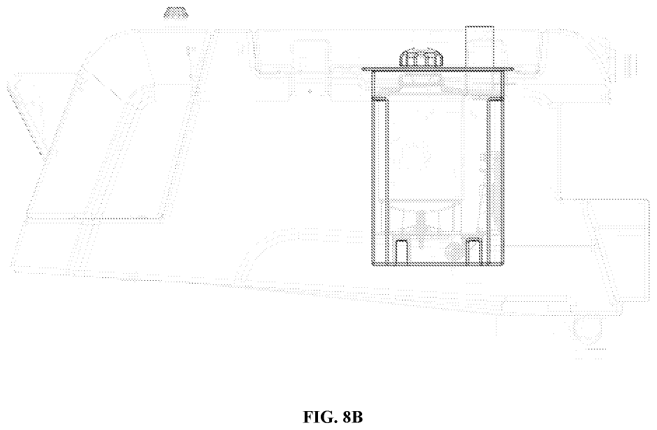

FIG. 8A is a perspective view of a semi-autonomous cleaning device displaying an internal disinfection module tank. FIG. 8B is a right-side view of a semi-autonomous cleaning device displaying an internal disinfection module tank. FIG. 9 is a top plan view of a semi-autonomous cleaning device displaying an external disinfection module tank.

FIG. 10 is a further perspective view of a semi-autonomous cleaning device with a revised external disinfection module. According to FIG. 10, semi-autonomous cleaning device 1000 includes a sprayer 1002, display 1004, tank 1006, clean water level indicator hose 1008, emergency stop button 1010 and pump 1012. Pump 1012 may be housed behind or enclosed inside tank 1006.

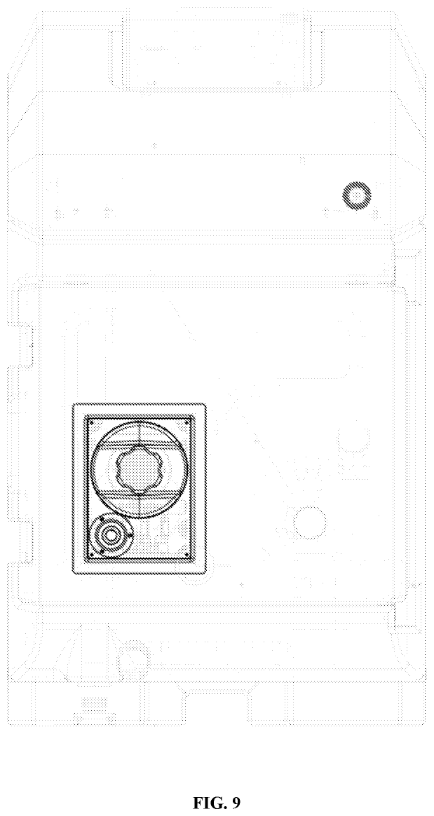

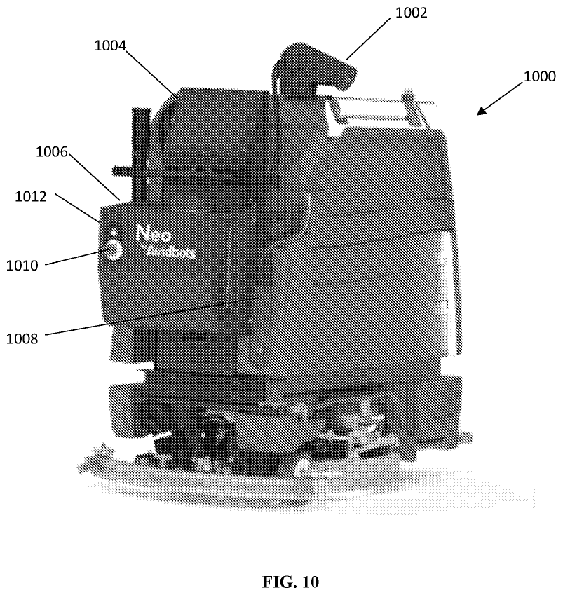

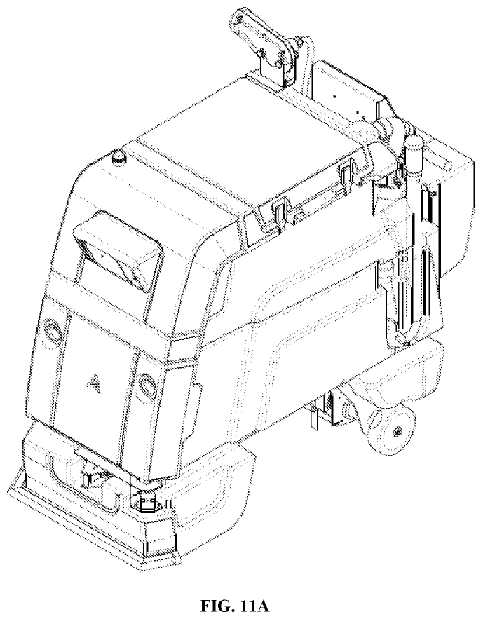

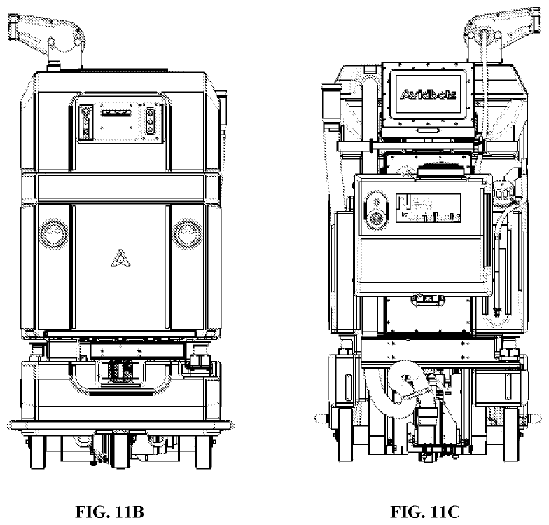

FIGS. 11A to 11E are line drawings illustrating a semi-autonomous cleaning device with another external disinfection module design. FIG. 11A is a front perspective view of a line drawing of a semi-autonomous cleaning device with an external disinfection module.

FIG. 11B is a front plan view of a line drawing of a semi-autonomous cleaning device with an external disinfection module. FIG. 11C is a back plan view of a line drawing of a semi-autonomous cleaning device with an external disinfection module.

FIG. 11D is a right-side view of a line drawing of a semi-autonomous cleaning device with an external disinfection module. FIG. 11E is a left side view of a line drawing of a semi-autonomous cleaning device with an external disinfection module.

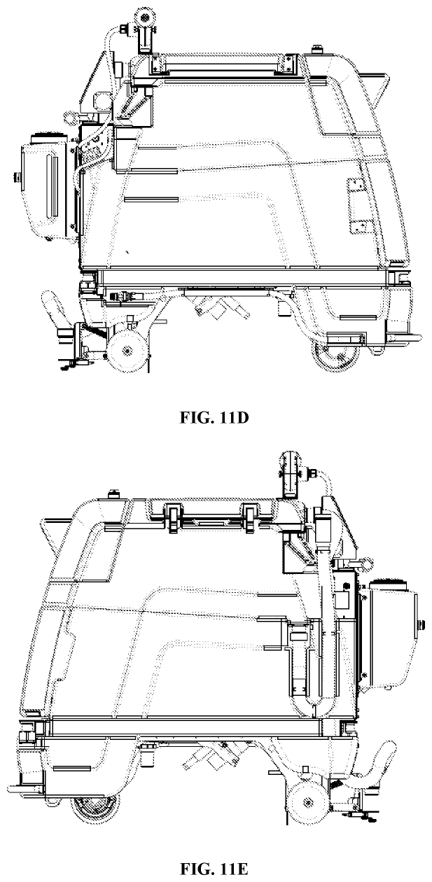

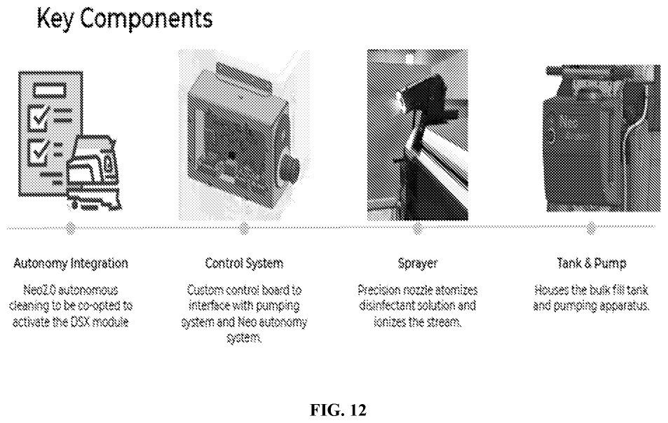

FIG. 12 is a diagram illustrating key components of the disinfection module. According to FIG. 12, key components of the disinfection module include:

### Autonomy Integration

Point to point navigation & disinfection
   Average disinfecting speed 0.6 m/s. This speed may be adjusted as required per location.
   Disinfection On/Off based on disinfection plan
   Obstacle avoidance and slow-downs/stop the spray
Seamless integration with robotic cleaning platform

### Control System

Input from autonomy system
Interfaces with Estop safety System
Custom printed circuit board assembly (PCBA)
Issues command to solution pump
   Powers electrostatic module
   Solution tank level detection

### Sprayer

Solution input from solution tank & pump
Houses replaceable atomization nozzle
Adjustable atomization nozzle (different apertures available)
LEDs mounted to spray nozzle illuminate target for safety
Adjustable spray angle

### Pump and Tank

Solution Tank
   Custom mounting bracket
   Wide fill cap with breather vent
   Digital level sensor
   Visual level check
Brushless motor pump
Clean out and shut off valve
Custom brackets and formed dust cover
Houses control system

According to FIG. 12, a semi-autonomous cleaning device is equipped with a disinfection module. This disinfection module will output a disinfectant spray with an illuminated target area (i.e., lighting showing spray pattern). The nozzle on the disinfection module can be customized to output a “fan” type spray pattern. This pattern can be configured to be a narrow to wide spray pattern (i.e., narrow or wide aperture selection).

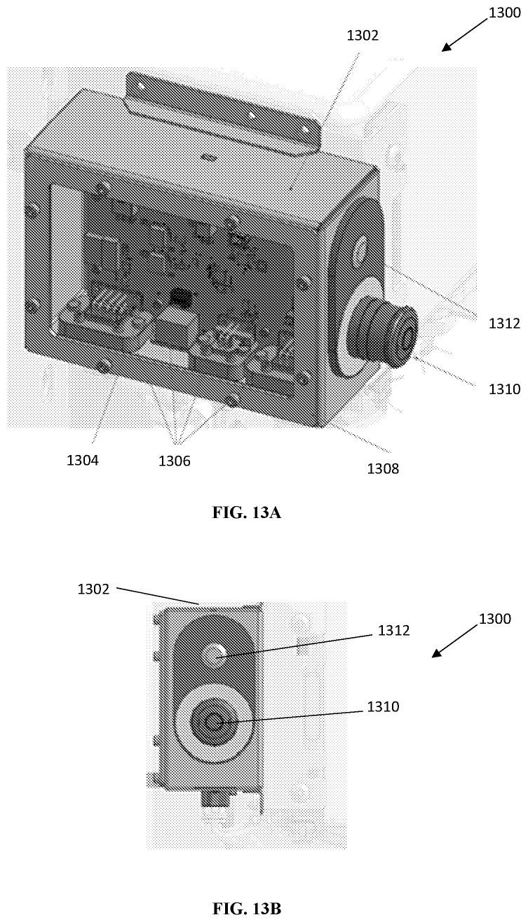

FIGS. 13A and 13B are diagrams illustrating the control system. According to FIG. 13A, control system 1300 is designed to integrate the solution tank (i.e., 3 gallon tank), pump system, control system and safety system with a vacuum formed dust cover enclosure 1302. Control system 1300 further consists of custom printed circuit board assembly (PCBA) 1304, inputs from autonomy system 1306 and outputs to pump and spray 1306, water-resistant gasket 1308, emergency stop button 1310 and semi-autonomous device main power button 1312.

As seen in FIG. 11 and FIG. 13B, control system 1300 is mounted vertically to fit snuggly with tank 1106. FIG. 13B is a right-side view of the control system. According to FIG. 13B, control system 1300 includes vacuum formed dust cover enclosure 1302, input/output connections 1312 and emergency stop button 1310. Emergency stop button 1310 may include a large red and yellow button that is easily visible.

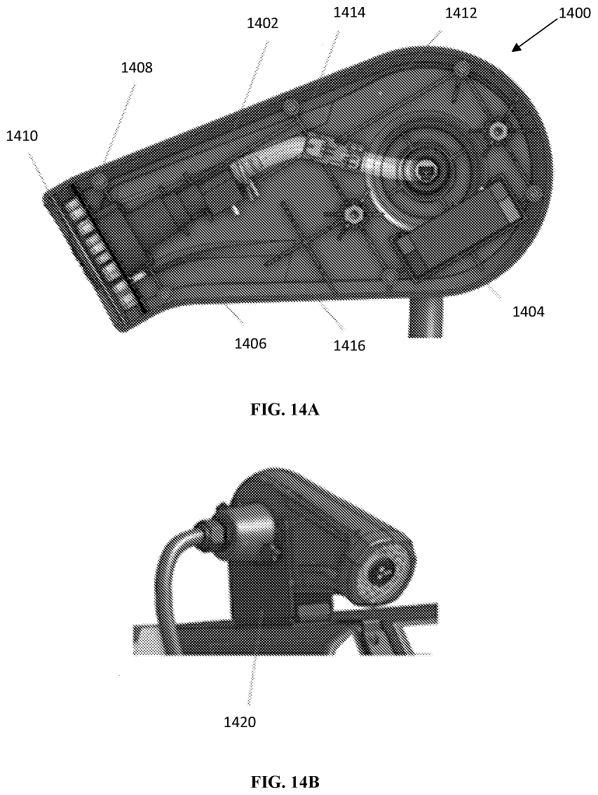

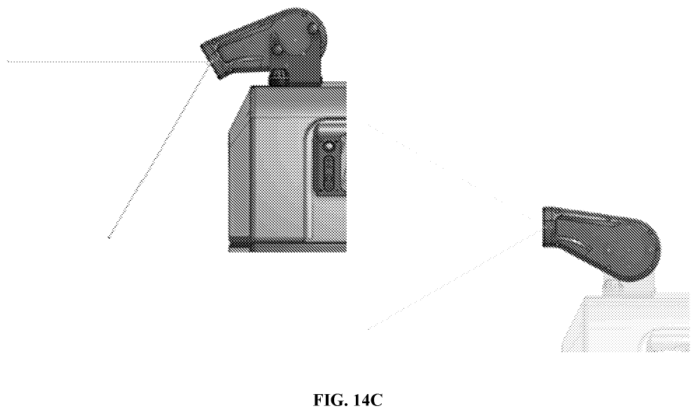

FIGS. 14A-14C are diagrams illustrating the sprayer module. According to FIG. 14A, sprayer module 1400 consists of a custom enclosure 1402 houses an electrostatic charge generation module 1404 where a nozzle 1406 atomizes electrostatically charged liquid disinfectant. The nozzle 1406 may be a replaceable nozzle with 3 positions. A LED spotlight 1408 and lens 1410 illuminates spray and target for safety and visual confirmation that the spray is operational. Sprayer module 1400 further includes a power distribution module 1412, a solution hose 1414 and an electrode module 1416. The electrode in the electrode module 1416 passes the electrostatic charge generated by 1404 to the fluid at the entry of the nozzle.

FIG. 14B illustrates sprayer module mounted on mounting bracket 1420 with user configurable variable spraying angle. FIG. 14C illustrates that the sprayer module may be manually adjusted to a user selectable spray angle.

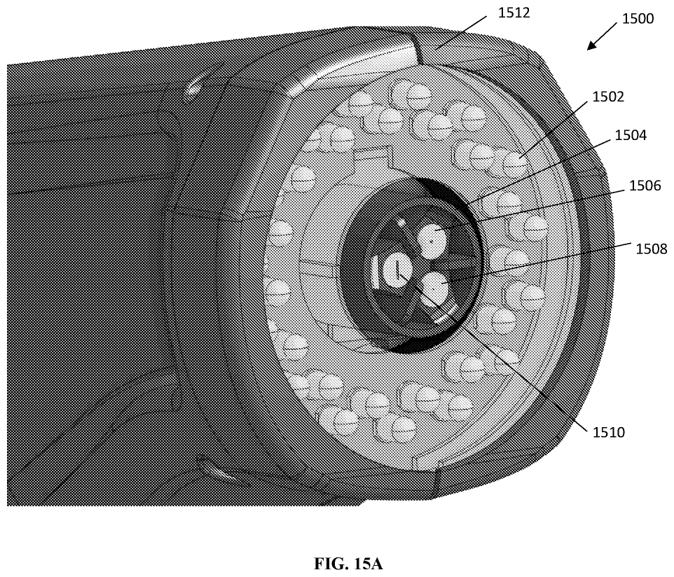

FIGS. 15A and 15B are diagrams illustrating the sprayer head of the sprayer module. FIG. 15A is a perspective view of the sprayer head of the sprayer module. FIG. 15B is a front plan view of the sprayer head of the sprayer module. According to FIGS. 15A and 15B, sprayer head assembly 1500 includes spray head cover 1512, a plurality of spray illumination light emitting diodes (LEDs) 1502.

Sprayer head assembly 1500 also includes spray housing nozzle housing 1504 that contains 3 nozzle head selections (1506, 1508, 1510). Nozzle head selections include a wide cone spray nozzle 1506, a narrow cone spray nozzle 1508 and a fan spray nozzle 1510. According to FIG. 15B, sprayer head assembly 1500 further includes adjustment knobs 1514 enabling the sprayer module to be adjusted to different spray angles.

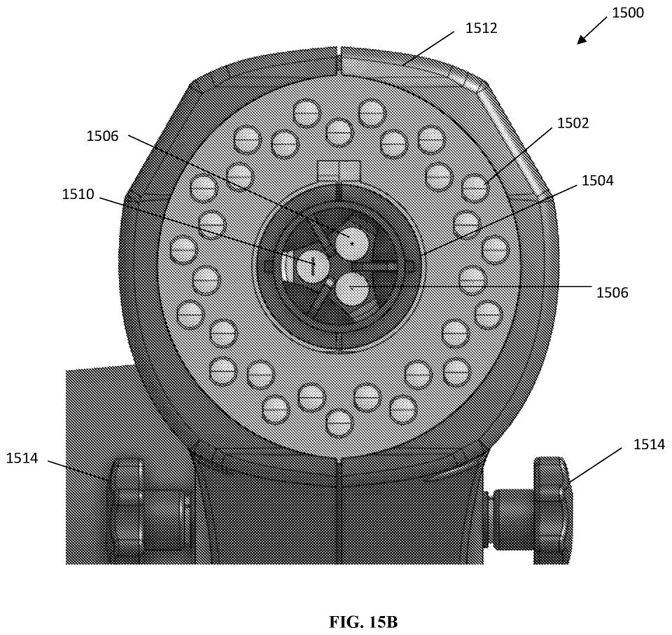

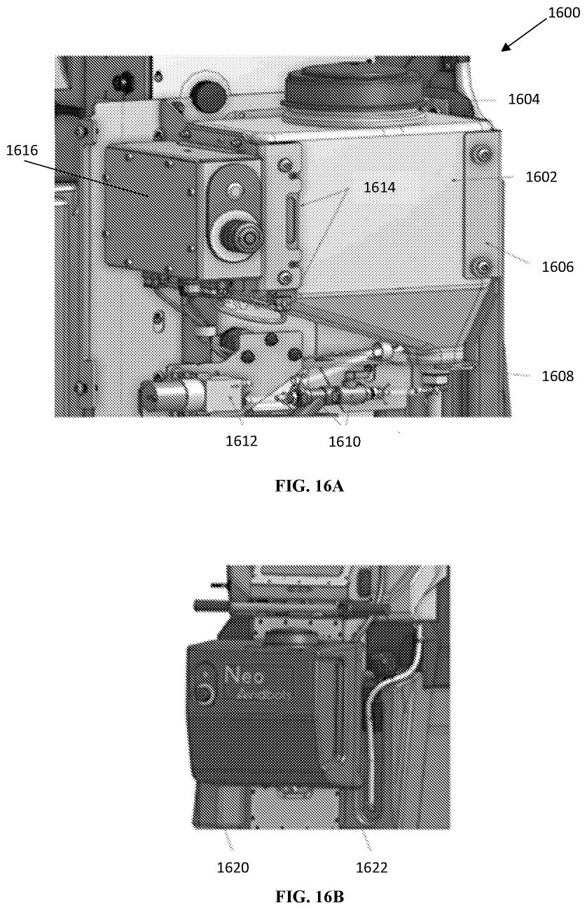

FIGS. 16A and 16B are diagrams illustrating the tank and pump module. According to FIG. 11 and FIGS. 16A and 16B, a semi-autonomous cleaning device 1100 is designed to integrate a tank and pump module 1600, including a plastic solution tank 1602, a pump system 1612, a control system 1616 and safety system 1010 (i.e., emergency stop button) with a vacuum formed dust cover 1620. Solution tank 1620 may include a 3 gallon tank, but other volume tank sizes can also be contemplated. Pump system 1612 includes a brushless pump, but may include other pump types.

According to FIG. 16A, tank and pump module 1600, further includes a wide mouth breather cap 1604, custom mounting bracket 1606, level sensors 1614 and DC pump 1612 (i.e., brushless pump). DC pump 1612 is connected to clean out and shut off valve 1610. Tank and pump module 1600 also includes an output hose 1608 that connects to a sprayer (not shown).

According to FIG. 16B, custom dust cover 1620 is shown as a cover protecting all the components of tank and pump module 1600. Custom dust cover 1620 also has a visual level indicator 1622.

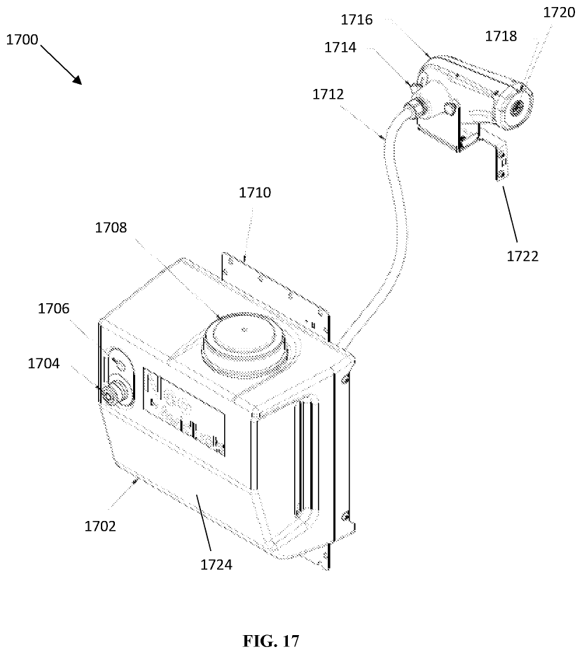

FIG. 17 is a line drawing illustrating a perspective view of components of a disinfection module. According to FIG. 17, disinfection module 1700, consists of fluid reservoir module 1702 connected to sprayer module 1716 by hose and wiring bundle 1712. Fluid reservoir module 1702 further comprises of power button 1706, e-stop (or emergency stop) button 1704, fill cap 1708, custom rear electrical enclosure panel 1710 and reservoir tank 1724. The capacity of reservoir tank 1724 has a fluid capacity of 3 gallons (11.4 liters).

According to FIG. 17, fluid reservoir module 1702 has a weight of 40 pounds (lbs) or 18 kilograms (kg) without fluid. In one embodiment, fluid reservoir module 1702 has a maximum power of 1 Amp at 36 Volts DC nominal. Fluid reservoir module 1702 also encompasses components of electrical enclosure panel 1710 including networking components such as 10/100 Ethernet and wireless connectivity options (i.e., WiFi® and Bluetooth®).

According to FIG. 17, sprayer module 1716, further comprises spray angle adjustment thumbscrews 1714, spray nozzle 1720 and indicator LED 1718. Sprayer module 1716 also includes mount 1722 configured to mount to the housing or frame of an autonomous or semi-autonomous cleaning device. Sensors may be placed to encode the spray angle. The spray angle data may be used in combination with route data, flow rate data, and site feature data to generate an effective mapping of the parts of surfaces that have been sanitized.

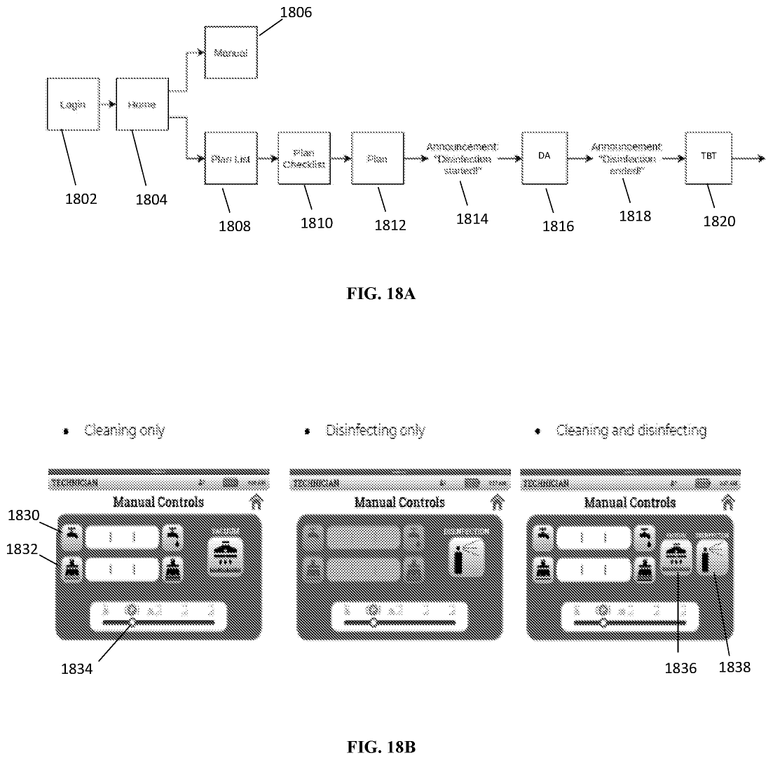

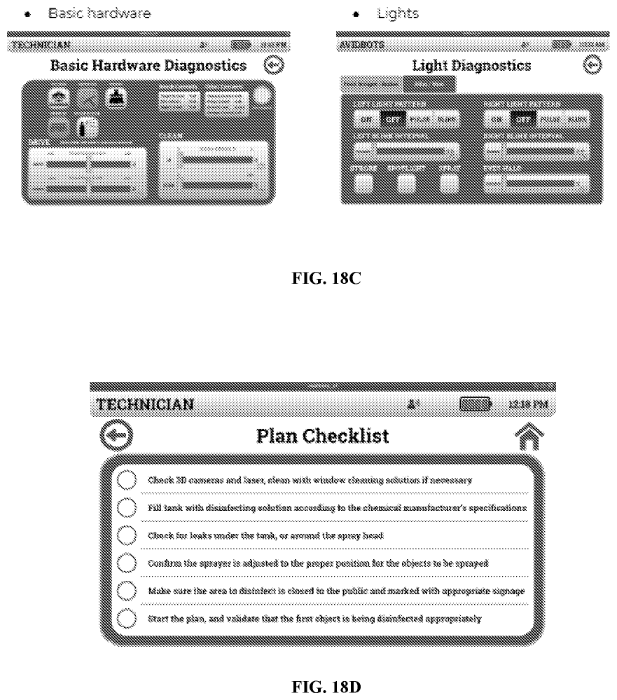

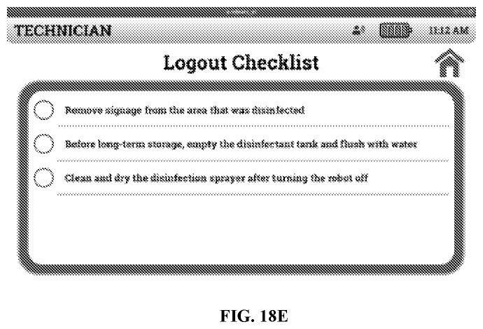

FIGS. 18A to 18E are diagrams illustrating graphical user interfaces (GUI) of the disinfection module. FIG. 18A is a block diagram illustrating the various GUI screens of the disinfection module. According to FIG. 18A, the first GUI screen is the Login screen 1802. Once logged in, the system goes to the Home screen 1804. The user has a choice of Manual operation 1806 or a Plan List 1808 operation.

If the user selects the Plan List 1808 is selected, the GUI moves onto Plan Checklist 1810 and then a Plan screen 1812. A notification 1814 will provide “Disinfection started” announcement. The GUI then moves to DA screen 1816, indicating that the system is “Disinfecting Autonomously”. Once the disinfection is completed, a further announcement 1818 of “Disinfection ended” is provided. Finally, the system will move to the TBT screen 1820, which represents “Travel between targets” in which the system will travel to another target and allow the system to reinitiate another plan 1808.

FIG. 18B illustrates a further GUI for “Cleaning only”, “Disinfecting only” and “Cleaning and disinfecting” user interfaces. According to FIG. 18B, touchscreen controls are provided for water level 1830, cleaning head pressure 1832, and direction and speed icon 1834 of the device. The direction and speed icon 1834 includes such selections as “reverse”, “park”, “speed 1”, “speed 2” and “speed 3”. Furthermore, button selections Vacuum 1836 and/or Disinfection 1838 icons are also provided.

FIG. 18C illustrates a further GUI for “Basic hardware” and “Lights” user interfaces. The “Basic hardware” user interface provides a menu and options for basic hardware diagnostics. The “Lights” user interface provides options for light diagnostics.

FIG. 18D illustrates a further GUI for a “Plan Checklist”. According to FIG. 18D, the “Plan Checklist” user interface provides different selection options for a plan checklist.

FIG. 18E illustrates a further GUI for a “Logout Checklist”. According to FIG. 18E, the “Logout Checklist” user interface provides different selection options for a logout checklist.

According to this disclosure, once a semi-autonomous cleaning device is equipped with a disinfection module, it provides facilities and operation executives with a solution that provides the following benefits: it cleans and sanitizes floors, disinfects high-touch areas and reports on cleaning and disinfection operations.

The solution reduces human uncertainty and cost, while remaining flexible and quick to deploy. It allows for using preferred chemicals and fine-tuning your processes and provides reports ensuring your service meets the expected level of quality and regulations.

The short-term impact is to enable operations of large public spaces (hospitals, airports, schools, train stations, office building, shopping centers, etc.) while controlling the spread of COVID-19. The longer-term impact is to ensure those places are safer against future viral or bacteriological outbreaks. We see this project as a long-term risk mitigation for the global economy.

According to this disclosure, the system has different use cases, including the following scenarios.

### Scenario 1—Continuous Spraying:

A cleaner or a cleaning supervisor would need to be able to use a semi-autonomous cleaning device to spray disinfecting chemicals on objects in large spaces (i.e., canteens, cafeterias, food courts, etc.). The space is expected to be emptied of people, prepared, and would need to be cleaned manually separately of food and garbage items. The cleaning device could spray in areas with carpet or hard floor.

The cleaning device should spray disinfectant continuously on objects all along its path, without distinction of objects/ON-OFF controls. The expectation is that objects at hand reach to be covered with disinfecting chemicals. A spraying coverage of 20 cm to 1.5 m from the robot, at tables height, would be appropriate in one embodiment. It needs to be done in a similar time-frame as what a regular cleaner would spend for a similar task.

In further embodiments, there is the ability to combine floor cleaning and spraying in the same run. The cleaning device can carry 2 different missions (i.e., cleaning plan and disinfection plan).

### Scenario 2—Continuous Spraying Up-Down:

A cleaner or a cleaning supervisor would need to be able to use the semi-autonomous cleaning device to spray disinfecting chemicals in open spaces from 8 ft. in height to the ground. The space is expected to be emptied of people and prepared, access forbidden during spraying and for 5 minutes after spraying for the solution to evaporate or settle. The robot could spray in areas with carpet or hard floor.

The robot should spray disinfectant continuously on walls and objects along its path, without distinction or ON-OFF controls. A spraying coverage of 20 cm to 1.5 m from the cleaning device, at tables height, would be appropriate. The walls need to be fully covered from 8 ft. high down to the ground. It needs to be done in a similar time-frame as what a regular cleaner would spend for a similar task. In one embodiment, the robot can carry solution for a runtime of 1 hour under those conditions.

If there is no intent to combine floor cleaning and spraying in the same run; the cleaning device can carry 2 different missions (cleaning plan and disinfection plan). The two mission strategy may be advantageous if it is undesirable to leave traces of disinfectant on the floor.

### Scenario 3—Disinfecting Touch Points:

A cleaner or a cleaning supervisor would need to be able to use the semi-autonomous cleaning device to spray disinfecting chemicals on touch areas, once a week to several times a day depending on the traffic in the area. Those touch surfaces include doorknobs, handles, touch screens, elevator buttons, tables, desks, ramps, vending machines. Some of those surfaces are not vertical, which could mean a need to change the angle of the spray if the divergence is not wide enough, but most are at the same height.

In addition, spraying people or sensitive surfaces can bring extra danger as disinfecting chemicals may be dangerous to inhale; consequently, there is a need to control where the spraying happens, an “always-on” spraying bringing way too many risks in this case. It is acceptable to have to isolate the area temporarily for the robot to operate, although people may still be present at 1 meter from the robot in areas it should not spray (example: spraying touchscreens in airports, in an area temporarily closed with stanchions). The robot could spray in areas with carpet or hard floor.

Touch areas to disinfect should be completely covered with disinfectant after the pass. A separate manual cleaning may still be required in cases where the disinfected surfaces get dirty after being touched. A cleaner may be expected to prepare some of the areas, for example, orientation of furniture and removal of debris. As such, in many environments, the spraying is preferably to be done at the same time as a person carrying complementary tasks in the same location as the robot.

Furthermore, the cleaning device must be able to spray doorknobs while remaining at least 1 m away for fire safety reasons. Some doors open out and can trigger the side safety stop, block the robot and block the people in the rooms, including during emergency evacuation. A spray of doors from 1 meter-1.5 meter will be appropriate in some embodiments.

According to this disclosure, a semi-autonomous cleaning device with a disinfection module would provide:
Targeted electrostatic disinfectant spraying or misting.
The proposed solution targets common touchpoints and high transmission points.
Use of existing semi-autonomous device and cleaning platform. Developing an add-on module which uses proven mapping technology to target high traffic areas. The system activates an electrostatically charged disinfection misting system which is designed to disinfect vertical surfaces (doorknobs, light switches, etc.) and select horizontal surfaces (backs of chairs, high set counters, etc.).

According to this disclosure, some difference with the proposed disclosure and other solutions include:
Other cleaning devices/robots do not combine both cleaning (scrubbers) and disinfection systems.
Other solutions only disinfect and bring little to no long-term return on investment (ROI); they provide no flexibility in the planning of using the product (scrubbing vs. disinfection).
Other solution may fog the site blindly; although not its only use case, our solution will be programmable to autonomously disinfect only certain surfaces in the location, on a map.
This solution differentiates itself from others in that it is designed specifically to target high viral transmission points. This is archived by hardware design and integration with an autonomous robotic platform.
In one embodiment, a targeted disinfection misting solution (hardware solution) is married to an autonomous robotic platform (robotic solution).

In further embodiments, the semi-autonomous cleaning device may include the following additional features:
A top mounted disinfection misting system.
A pump to push disinfectant solution through a misting nozzle.
A small fan creates a powerful air current that the mist is deposited into.
The solution is electrostatically charged allowing it to cling to surfaces, reach hidden surfaces, and improving overall coverage.
The pump, power controls and solution tank to reside internally inside the dirty water tank of the semi-autonomous cleaning device platform.
The logic control to reside inside the device nose cone.

According to other embodiments, the proposed solution consists of an autonomous and/or semi-autonomous cleaning device that focuses on the combination of spraying, spraying under autonomous control, and use of an autonomous robotic platform that provides a targeted disinfection of touchpoints.

In further embodiments, the autonomous software of the semi-autonomous cleaning device is updated to include “disinfection zones” so the device can trigger the disinfectant module when the robot is close to an area of interest. The device will also require a planning “route” to navigate through a series of points as required for disinfection.

The functions described herein may be stored as one or more instructions on a processor-readable or computer-readable medium. The term “computer-readable medium” refers to any available medium that can be accessed by a computer or processor. By way of example, and not limitation, such a medium may comprise RAM, ROM, EEPROM, flash memory, CD-ROM or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor. A “module” can be considered as a processor executing computer-readable code.

A processor as described herein can be a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but in the alternative, the processor can be a controller, or microcontroller, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, any of the signal processing algorithms described herein may be implemented in analog circuitry. In some embodiments, a processor can be a graphics processing unit (GPU). The parallel processing capabilities of GPUs can reduce the amount of time for training and using neural networks (and other machine learning models) compared to central processing units (CPUs). In some embodiments, a processor can be an ASIC including dedicated machine learning circuitry custom-build for one or both of model training and model inference.

The disclosed or illustrated tasks can be distributed across multiple processors or computing devices of a computer system, including computing devices that are geographically distributed.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

While the foregoing written description of the system enables one of ordinary skill to make and use what is considered presently to be the best mode thereof, those of ordinary skill will understand and appreciate the existence of variations, combinations, and equivalents of the specific embodiment, method, and examples herein. The system should therefore not be limited by the above described embodiment, method, and examples, but by all embodiments and methods within the scope and spirit of the system. Thus, the present disclosure is not intended to be limited to the implementations shown herein but is to be accorded the widest scope consistent with the principles and novel features disclosed herein.

1. A semi-autonomous cleaning apparatus for cleaning surfaces, comprising:
a frame supporting at least one storage volume;
a drive system supported by the frame and configured to move the frame along a surface;
a cleaning assembly coupled to the frame and configured to transfer debris from the surface to the at least one storage volume as the drive system moves the cleaning assembly along the surface;
a front or rear sensing module comprising a plurality of sensors capable of computing and guiding the path and direction of the cleaning apparatus;
a disinfection module to spray a disinfectant solution; and
an electronics system supported by the frame and including at least a memory and a processor, the processor being configured to execute a set of instructions stored in the memory and receiving input from the front and rear sensing module to perform at least one or more actions in relations to the disinfection module.

2. The semi-autonomous cleaning apparatus of claim 1 wherein the disinfection module further comprising a fan, an atomizer nozzle, and an electrostatic module.

3. The semi-autonomous cleaning apparatus of claim 2 wherein the fan is a high powered DC fan.

4. The semi-autonomous cleaning apparatus of claim 1 wherein the disinfection solution is selected from a list consisting of quaternary ammonium, hydrogen peroxide and iso-propyl.

5. The semi-autonomous cleaning apparatus of claim 1 wherein the distance between the atomizer nozzle and a vertical surface to be disinfected is controlled during disinfection.

6. The semi-autonomous cleaning apparatus of claim 1 wherein the locations and surfaces within a facility that have been disinfected are logged.

7. The semi-autonomous cleaning apparatus of claim 1 wherein the locations and surfaces within a facility that have been disinfected are mapped onto a representation of the facility.

8. A disinfection apparatus, configured to spray a disinfection solution, on a semi-autonomous cleaning apparatus, the disinfection apparatus comprising:
a control system;
a sprayer module;
a storage tank to store a disinfection solution; and
a pump system configured to pump the disinfection module to be dispelled through the sprayer module;
wherein the sprayer module is adjustably mounted on the semi-autonomous cleaning apparatus;
wherein the sprayer module is configured to spray the disinfection solution at different spray angles.

9. The disinfection apparatus of claim 8 wherein the sprayer module further comprises a plurality of LED lights, a sprayer head assembly, an adjustment knob, a power distribution module, a solution hose and an electrode module.

10. The disinfection apparatus of claim 8 wherein the sprayer module further comprises a spray housing nozzle having at least 3 nozzle heads.

11. The disinfection apparatus of claim 11 wherein the nozzle heads is selected from a list consisting of a wide cone spray nozzle, a narrow cone spray nozzle, and a fan spray nozzle.

12. The disinfection apparatus of claim 8 further comprising a power distribution module, a plastic solution tank, a dust cover, a power button, a safety system, an electrical panel, a circuit board.

13. The disinfection apparatus of claim 12 wherein the safety system further comprises an emergency stop button.

14. The disinfection apparatus of claim 12 wherein the dust cover further comprises a visual level indicator.

15. The disinfection apparatus of claim 8 wherein the pump system further comprises a wide mouth breather cap, custom mounting brackets, level sensors and a pump.

16. The semi-autonomous cleaning apparatus of claim 1 further comprising an electronic display configured to show a graphical user interface (GUI) of the different operating modes of the disinfection module.

17. The semi-autonomous cleaning apparatus of claim 16 wherein the different operating modes is selected from a list consisting of Cleaning Only, Disinfecting Only, Cleaning and Disinfecting, Basic Hardware diagnostics and Light diagnostics.

18. The semi-autonomous cleaning apparatus of claim 1 wherein the disinfection module further comprises a sprayer module, a storage tank to store a disinfection solution, and a pump system configured to pump the disinfection module to be dispelled through the sprayer module.

19. The semi-autonomous cleaning apparatus of claim 18 wherein the sprayer module is adjustably mounted on the semi-autonomous cleaning apparatus.

20. A method of operating a disinfection module on a semi-autonomous cleaning device, the method comprising:
logging into the semi-autonomous cleaning device;
displaying a home screen graphical user interface screen;
displaying a choice of a manual operation or Plan List operation mode;
if the Plan List operation mode is selected;
displaying a Plan Checklist GUI screen;
selecting an operating Plan;
providing a notification message of “Disinfection Started”;
initiating the disinfection plan; and
providing a notification message of “Disinfection ended” once the plan has been completed.
